(19) United States
(12) Patent Application Publication
(10) Pub. No.: US 2024/0216653 A1
(43) Pub. Date: Jul. 4, 2024

(54) INTRAVASCULAR DEVICE WITH A MATCHED DIAMETER CORE WIRE JOINT

(71) Applicant: SCIENTIA VASCULAR, INC., West Valley City, UT (US)

(72) Inventors: Brandon Rodney POPE, Salt Lake City, UT (US); William Rigby ECCLES, Centerville, UT (US); John A. LIPPERT, Park City, UT (US); Edward J. SNYDER, Park City, UT (US)

(21) Appl. No.: 18/391,432

(22) Filed: Dec. 20, 2023

Related U.S. Application Data
(60) Provisional application No. 63/434,438, filed on Dec. 21, 2022.

(51) Int. Cl. A61M 25/09

(52) U.S. Cl. CPC A61M 25/09; A61M 2025/09091; A61M 2025/09133; A61M 2025/09166; A61M 2025/09175; A61M 2205/0216

(57) ABSTRACT

Disclosed are intravascular devices comprising a core having a proximal and distal section and a tube wherein the distal section of the core passes into and is encompassed by the tube. The core includes a stepped shoulder located at the junction of the proximal and distal sections of the core. The proximal end of the tube is coupled to the stepped shoulder beneficially increasing torque transmission along the tube to the distal end of the intravascular device while maintaining effective flexibility of the device.

### CROSS-REFERENCE TO RELATED APPLICATIONS

This application claims the benefit of and priority to U.S. Provisional Patent Application No. 63/434,438, filed Dec. 21, 2022 and titled “INTRAVASCULAR DEVICE WITH A MATCHED DIAMETER CORE WIRE JOINT,” the entirety of which is incorporated herein by reference.

### BACKGROUND

Guidewire devices are often used to lead or guide catheters or other interventional devices to a targeted anatomical location within a patient's body. Typically, guidewires are passed into and through a patient's vasculature in order to reach the target location, which may be at or near the patient's heart or brain, for example. Radiographic imaging is typically utilized to assist in navigating a guidewire to the targeted location. In many instances, a guidewire is placed within the body during the interventional procedure where it can be used to guide multiple catheters or other interventional devices to the targeted anatomical location.

Some guidewire devices are constructed with a curved or bent tip to enable an operator to better navigate a patient's vasculature. With such guidewires, an operator can apply a torque to the proximal end of the guidewire or attached proximal handle in order to orient and point the tip in a desired direction. The operator may then direct the guidewire further within the patient's vasculature in the desired direction.

Tuning the flexibility of a guidewire device, particularly the distal sections of the guidewire device, is also a concern. In many circumstances, relatively high levels of flexibility are desirable in order to provide sufficient bendability of the guidewire to enable the guidewire to be angled through the tortuous bends and curves of a vasculature passageway to arrive at the targeted area. For example, directing a guidewire to portions of the neurovasculature requires passage of the guidewire through curved passages such as the carotid siphon and other tortuous paths.

Another concern related to guidewire devices is the ability of a given guidewire device to transmit torque from the proximal section to the distal section (i.e., the “torque transmission,” or “torquability,” of the guidewire device). As more of a guidewire is passed into and through a tortuous vasculature passageway, the amount of frictional surface contact between the guidewire and the vasculature increases, hindering easy movement of the guidewire through the vasculature passage. A guidewire with good torquability enables torsional forces at the proximal end to be transmitted through the guidewire to the distal end so that the guidewire can rotate and overcome the frictional forces.

Some guidewire devices include a distally placed micro-machined hypotube positioned over the distal end of the guidewire core in order to direct applied torsional forces further distally toward the end of the device. Because torsional forces are primarily transmitted through the outer sections of a cross-section of a member, the tube is configured to provide a path for increased transmission of torque as compared to the amount of torque transmitted by a guidewire core not sheathed by a tube. Typically, such tubes are formed from a super-elastic material such as nitinol so as to provide desired torque transmission characteristics in addition to providing good levels of flexibility.

Guidewires are available with various outer diameter sizes. Widely utilized sizes include 0.010, 0.014, 0.016, 0.018, 0.024, 0.035, and 0.038 inches, for example, though they may also be smaller or larger in diameter. Because torque transmission is a function of diameter, larger diameter guidewires typically have greater torque transmission. On the other hand, smaller diameter guidewires typically have greater flexibility.

A catheter used in conjunction with a guidewire will be sized with an inner diameter somewhat larger than the outer diameter of the guidewire to enable the catheter to be positioned over and translated upon the guidewire. The difference in size between the guidewire and catheter can affect the ability of the catheter to travel along the guidewire. For example, the larger the annular space between the outer diameter of the guidewire and the inner diameter of the catheter, the greater the amount of radial play the catheter may experience and the more difficult it may be to navigate the catheter over the guidewire. With excessive radial play, the distal end of the catheter may have a higher risk of catching against vasculature or other anatomy of the patient rather than smoothly following along the guidewire path.

While such guidewires have seen success, several limitations remain. In particular, torque transmission through the tube to the distal end of the guidewire is less than desired, especially for smaller-diameter guidewires. Also, variations in the diameter of the guidewire resulting from coupling the tube to the core increase the annular space between the guidewire and the catheter at the attachment point of the tube and core and thus hinder effective intravascular navigation.

### SUMMARY

Disclosed herein is an intravascular device including: a core having a proximal section and a distal section, wherein the core includes a stepped shoulder at a transition from the proximal section to the distal section, the stepped shoulder having a constant outer diameter, the core forming a distally facing surface proximal of the stepped shoulder; and a tube coupled to the core at the stepped shoulder, the distal section of the core passing into and being encompassed by the tube.

The outer diameter of the stepped shoulder and an inner diameter of the tube can be substantially similar. An outer diameter of the tube can be substantially the same as an outer diameter of the proximal section of the core. The tube can abut the distally facing surface. The stepped shoulder can have an outer diameter that is 30% to 90% of the outer diameter of the proximal section of the core.

The stepped shoulder can have a length of about 1 cm to about 20 cm, or about 1 cm to about 10 cm, or about 1 cm to about 5 cm, or about 1 cm to about 2 cm, or be within a range using any combination of the foregoing as endpoints.

In one embodiment, an intravascular device includes: a core having a proximal section and a distal section, wherein the core includes a stepped shoulder at a transition from the proximal section to the distal section, the stepped shoulder having a constant outer diameter, the core forming a distally facing surface adjacent to and proximal of the stepped shoulder, wherein the distally facing surface is perpendicular to a longitudinal axis of the core; and a tube coupled to the core at the stepped C shoulder, the distal section of the core passing into and being encompassed by the tube, and the tube abutting the distally facing surface, wherein the outer diameter of the stepped shoulder and an inner diameter of the tube are substantially similar, wherein an outer diameter of the tube is substantially the same as an outer diameter of the proximal section of the core.

The configuration of the disclosed intravascular device can beneficially provide a relatively larger surface for bonding between the tube and the core, which can decrease the amount of adhesive needed to secure the tube to the core, increase the torque transmitted to the tube from the core, and remove the need for a bushing or other securement device.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1 illustrates an embodiment of a guidewire device having a core and a tube coupled to the distal section of the core.

FIG. 2 illustrates an embodiment of a core of a guidewire device, the core comprising a first tapered shoulder.

FIG. 3 illustrates an embodiment of a guidewire device having a core and a tube coupled to the distal section of the core, the tube and the core forming a neck.

FIG. 4 illustrates an embodiment of a guidewire device comprising a core with a stepped shoulder located at the transition between the proximal and distal sections of the core.

FIG. 5 illustrates an embodiment of a guidewire device comprising a core and a tube coupled to the distal section of the core, wherein the core includes a stepped shoulder located at the transition between the proximal and distal sections of the core, the inner diameter of the tube and the outer diameter of the stepped shoulder being substantially similar.

### DETAILED DESCRIPTION

### Overview of Guidewire Structural Features

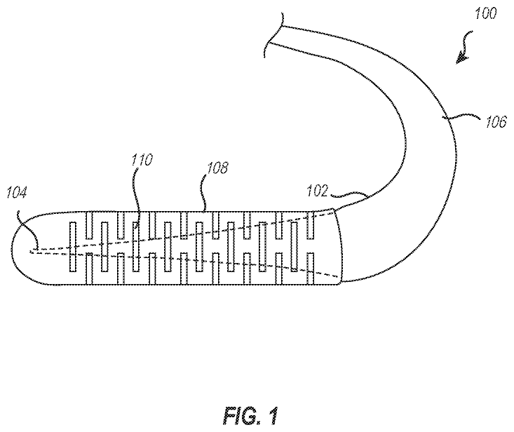

FIG. 1 schematically illustrates a guidewire 100 suitable for utilizing one or more features of the present disclosure. The illustrated guidewire 100 includes a core 102 and a tube 108. The core 102 includes a distal section 104 that extends into the tube 108 as shown. The distal section 104 may be tapered, either continuously or in one or more discrete sections, so that the more distal sections have a smaller diameter and greater flexibility than more proximal sections. For example, the distal section 104 may be ground so as to progressively taper to a smaller diameter at the distal end. In some embodiments, the distal section 104 may be flattened into a ribbon-like shape with a flat, rectangular, or oblong cross section.

The core 102 and the tube 108 are typically formed from different materials. For example, the tube 108 is preferably formed from a relatively flexible and elastic material such as nitinol, whereas the core 102 may be formed from a relatively less flexible and elastic material such as stainless steel. Forming the core 102 from stainless steel may be advantageous because it allows the distal tip to hold a shape when selectively bent/shaped by an operator and because stainless steel provides sufficient modulus of elasticity to provide more responsive translational movement. While these materials are presently preferred, other suitable materials such as polymers or other metals/alloys may also be utilized.

The tube 108 is coupled to the core 102 (e.g., using adhesive, soldering, and/or welding) in a manner that beneficially allows torsional forces to be transmitted from the core 102 to the tube 108 and thereby to be further transmitted distally by the tube 108. A medical grade adhesive or other suitable material may be used to couple the tube 108 to the core 102 at the distal end of the device to form an atraumatic covering.

The tube 108 may include a cut pattern that forms fenestrations 110 in the tube 108. The pattern of fenestrations 110 can include axially extending “beams” and circumferentially extending “rings” as shown, and/or may be arranged to provide desired flexibility characteristics to the tube 108, including the promotion of preferred bending directions, the reduction or elimination of preferred bending directions, or gradient increases in flexibility along the longitudinal axis, for example. Examples of cut patterns and other guidewire device features that may be utilized in the guidewire devices described herein are provided in detail in U.S. Pat. No. 11,369,351, which is incorporated herein by reference in its entirety. Other patterns of slots or fenestrations may additionally or alternatively be included.

The guidewire 100 typically has a length ranging from about 50 cm to about 350 cm depending on particular application needs. The tube 108 may have a length ranging from about 20 cm to about 65 cm, more typically about 30 cm to about 55 cm such as about 35 cm to about 45 cm.

### Core Wire and Tube Structure Connection

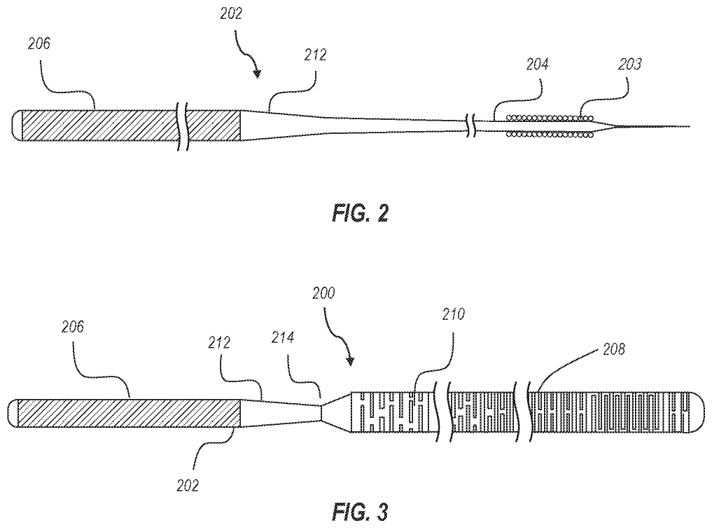

FIG. 2 illustrates the core 202 of a guidewire 200. The guidewire 200 may include any of the general features described above in relation to guidewire 100, with like reference numbers indicating like parts. The guidewire 200 may have a diameter of about 0.014 inches to about 0.035 inches, though larger or smaller sizes may also be utilized depending on particular application needs, and the features of the present disclosure are not necessarily limited to certain guidewire sizes. Some embodiments may have outer diameter sizes corresponding to standard guidewire sizes such as 0.014 inches, 0.016 inches, 0.018 inches, 0.024 inches, 0.035 inches, or other such sizes common to guidewire devices.

The distal section 204 of the core 202 may have a smaller diameter than the proximal section 206. The core 202 may taper beginning with a first tapered shoulder 212 and may continue to taper to a diameter of about 0.002 inches, or a diameter within a range of about 0.001 to 0.005 inches. Tapering core 202 increases the flexibility of the distal section 204 of the core 202, although because torque transmission is a function of outer diameter, significant torquability is lost. In some embodiments, the distal tip may be flattened (e.g., to a rectangular cross section) to further enhance bending flexibility while minimizing reductions in cross-sectional area needed for tensile strength. In such embodiments, the cross section may have dimensions of about 0.001 inches by 0.003 inches, for example.

The guidewire 200 may also include a coil 203 positioned upon at least a portion of the distal section 204 of the core 202. The coil 203 is preferably formed from one or more radiopaque materials, such as platinum group, gold, silver, palladium, iridium, osmium, tantalum, tungsten, bismuth, dysprosium, gadolinium, and the like. Additionally, or alternatively, the coil 203 may be at least partially formed from a stainless steel or other material capable of effectively holding shaped after being bent or otherwise manipulated by a user.

FIG. 3 illustrates the guidewire 200 including the core 202 of FIG. 2 and now showing a tube 208 (optionally with fenestrations 210) coupled to the distal section of the core 202. The tube 208 may have a length within a range of about 3 to 100 cm, for example. The proximal end of the tube 208 is coupled to the core 202 at the distal portion of the first tapered shoulder 212.

In some embodiments, the tube 208 has a uniform inner diameter. Because the distal section 204 of the core 202 tapers, contact between the inner surface of the tube 208 and the surface of the distal section 204 of the core 202 is minimal, resulting in a smaller surface area for adequate bonding of the tube 208 to the core 202. To ensure sufficient coupling of the proximal end of the tube 208 to the core 202, and thus ensure sufficient torque transmission to the tube 208, a relatively copious amount of adhesive, a bushing (to increase the surface area for binding), and/or other securement components are generally required.

As a result of the connection between the tube 208 and the core 202, a neck 214 is formed extending from the first tapered shoulder 212 of the core 202 to the proximal end of the tube 208. The neck 214 interferes with the smooth intravascular navigation of a catheter traveling along the guidewire 200. Excessive radial play between the catheter and the guidewire 200 will be present as the distal end of the catheter passes the neck 214, increasing the likelihood the catheter will catch on intravascular tissue. The neck 214 may also interfere with the ability of the guidewire 200 to smoothly translate along the patient's vasculature.

In some embodiments, the coil 203 has a length that substantially coincides with the length of the tube 208. In other embodiments, the coil 203 is shorter. Some embodiments may include two or more coils.

In some embodiments, the coil 203 (or a set of multiple coils) is sized to fill and pack the space between the distal section 204 of the core 202 and the tube 208. For example, the coil 203 (or a set of multiple coils) may be sized so as to abut both the core 202 and the inner surface of the tube 208. The coil 203 (or a set of multiple coils) may therefore function to pack the space between the core 202 and the tube 208 so as to align the curvature of the core 202 with the curvature of the tube 208.

For example, when a curvature is formed in the tube 208, the closely packed segments of the coil 203 (or a set of multiple coils) functions as a packing between the tube 208 and the core 202 to impart the same curvature to the core 202. In contrast, a guidewire device omitting such features would, when curved at the tube, not follow the same curve as the tube but would extend until abutting against the inner surface of the tube before being forced to curve. Aligning the curvature of the core 202 and the tube 208 can beneficially minimize the buildup of mismatched forces within the core 202 which, when released, can detrimentally cause the distal end of the device to “whip.” Such whipping can disrupt intended guidewire navigation or even damage sensitive anatomy.

Guidewire Device with a Stepped Shoulder

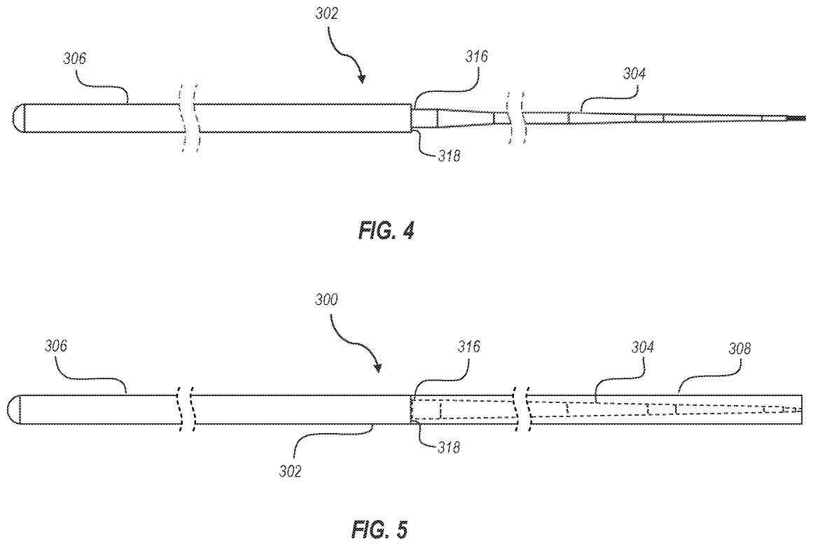

FIG. 4 illustrates an exemplary embodiment of a core 302 of a guidewire 300 (shown in FIG. 5). The guidewire 300 (including the core 302) may include any of the general features described above in relation to guidewires 100 and 200, with like reference numbers indicating like parts, except as noted. Unlike the guidewire 200 with tapered shoulder 212, the illustrated core 302 includes a stepped shoulder 316 at the transition between the proximal section 306 and the distal section 304 of the core 302. Associated with the stepped shoulder 316 is a distally facing surface 318 contiguous with the proximal section 306 of the core 302.

As shown, the outer surface of the core 302 at the stepped shoulder 316 extends substantially parallel to the longitudinal axis of the core 302, and the distally facing surface 318 is substantially perpendicular to the longitudinal axis of the core 302. This is in contrast to the tapered shoulder 212 of guidewire 200, which is not associated with a distally facing surface and which has an outer surface that extends at a transverse angle to form the taper.

The stepped shoulder 316 has an outer diameter that is smaller than the outer diameter of the proximal section 306 of the core. The length of the stepped shoulder 316 may be 1 cm to 20 cm, or 1 cm to 10 cm, or 1 cm to 5 cm, such as 2 cm, or a length within a range using any of the foregoing as endpoints.

The stepped shoulder 316 of the core 302 may be formed by cutting and/or grinding the distal section 304 of the core 302 to a profile. Alternatively, the core 302 may be formed by attaching a first elongated member (which forms proximal section 306) and a second elongated member (which forms distal section 304), the first elongated member having a larger diameter than the second elongated member, the proximal section of the second elongated member passing into and being encompassed by the distal section of the first elongated member, wherein the proximal end of the second elongated member and the distal end of the first elongated member form the stepped shoulder 316.

FIG. 5 illustrates an exemplary embodiment of guidewire 300 including the core 302 of FIG. 4 as well as tube 308. The guidewire 300 may also include a hydrophilic or other friction-reducing coating. The distal section 304 of the core 302 passes into and is encompassed by the tube 308. The inner diameter of the tube 308 and the outer diameter of the stepped shoulder 316 may be substantially similar to provide a close fit therebetween.

The proximal end of the tube 308 may be coupled to the core 302 at the stepped shoulder 316. The inner surface of the tube 308 and the surface of the stepped shoulder 316 may be in contact over substantially the entire length of the stepped shoulder 316. The surface of the stepped shoulder 316 presents a relatively larger surface to which the tube 308 may be bonded. This lager surface decreases the amount of adhesive needed to secure the tube 308 to the core 302, increases the torque transmitted to the tube 308 from the core 302, and removes the need for a bushing or other securement device.

The uniform diameter of the stepped shoulder 316 enables a tube 308 with a uniform inner diameter to slide along the stepped shoulder 316 until the proximal end of the tube 308 abuts against the distally facing surface 318. The tube 308, when so positioned, does not form a neck (i.e., eliminates a feature corresponding to neck 214) and thus minimizes the limitations of such a neck and therefore improves the intravascular navigation of a guidewire 300 or a catheter traveling along it.

The tube 308 may also have a substantially similar outer diameter as the outer diameter of the proximal section 306 of the core 302, such that the outer diameter of the guidewire 300 is substantially uniform over its full length. A substantially uniform diameter is beneficial to the operation of the guidewire 300 because it decreases or eliminates structure of the guidewire 300 on which intravascular tissue may catch and creates smoother translation of the device along the patient's vasculature.

A substantially uniform diameter is also beneficial when applying a coating to the guidewire 300. A substantially uniform diameter minimizes surface area irregularities of the guidewire 300 (and thus reducing the amount of coating needed to cover the device) and helps to ensure an even distribution of coating along the complete length of the guidewire 300.

The stepped shoulder 316 can have an outer diameter that is 30% to 90% of the outer diameter of the proximal section 306 of the core 302, such as 40% to 80%, or 50% to 70%, or 60% of the outer diameter of the proximal section 306 of the core 302, or be within a range using any combination of the foregoing as endpoints.

### EXAMPLE EMBODIMENTS

The following list of clauses represents a non-exhaustive list of example embodiments within the scope of the present disclosure.

Clause 1. An intravascular device, comprising: a core having a proximal section and a distal section, wherein the core includes a stepped shoulder at a transition from the proximal section to the distal section, the stepped shoulder having a constant outer diameter, the core forming a distally facing surface proximal of the stepped shoulder; and a tube coupled to the core at the stepped shoulder, the distal section of the core passing into and being encompassed by the tube.

Clause 2. The intravascular device of clause 1, wherein the outer diameter of the stepped shoulder and an inner diameter of the tube are substantially similar.

Clause 3. The intravascular device of clause 1 or clause 2, wherein an outer diameter of the tube is substantially the same as an outer diameter of the proximal section of the core.

Clause 4. The intravascular device of clause 1 or clause 2, wherein an outer diameter of the tube is greater than an outer diameter of the proximal section of the core.

Clause 5. The intravascular device of any preceding clause, the device further comprising a hydrophilic coating.

Clause 6. The intravascular device of any preceding clause, wherein the tube is formed from a super-elastic material.

Clause 7. The intravascular device of any preceding clause, wherein at least a portion of the distal section of the core is a flat ribbon.

Clause 8. The intravascular device of any preceding clause, wherein the core is formed from stainless steel.

Clause 9. The intravascular device of any preceding clause, further comprising a radiopaque element disposed at or near a distal end of the device.

Clause 10. The intravascular device of any preceding clause, wherein the tube includes a plurality of fenestrations.

Clause 11. The intravascular device of clause 10, wherein the plurality of fenestrations form a plurality of axially extending beams and a plurality of circumferentially extending rings.

Clause 12. The intravascular device of any preceding clause, wherein the stepped shoulder has a length of about 1 cm to about 20 cm, or about 1 cm to about 10 cm, or about 1 cm to about 5 cm, or about 1 cm to about 2 cm.

Clause 13. The intravascular device of any preceding clause, wherein the distally facing surface is perpendicular to a longitudinal axis of the core.

Clause 14. The intravascular device of any preceding clause, wherein the tube abuts the distally facing surface.

Clause 15. The intravascular device of any preceding clause, wherein the distal section of the core further comprises one or more tapered shoulders distal of the stepped shoulder.

Clause 16. The intravascular device of any preceding clause, further comprising one or more coils disposed between the core and the tube.

Clause 17. The intravascular device of any preceding clause, wherein the stepped shoulder has an outer diameter that is 30% to 90% of the outer diameter of the proximal section of the core.

Clause 18. An intravascular device, comprising: a core having a proximal section and a distal section, wherein the core includes a stepped shoulder at a transition from the proximal section to the distal section, the stepped shoulder having a constant outer diameter, the core forming a distally facing surface adjacent to and proximal of the stepped shoulder, wherein the distally facing surface is perpendicular to a longitudinal axis of the core; and a tube coupled to the core at the stepped shoulder, the distal section of the core passing into and being encompassed by the tube, and the tube abutting the distally facing surface, wherein the outer diameter of the stepped shoulder and an inner diameter of the tube are substantially similar, wherein an outer diameter of the tube is substantially the same as an outer diameter of the proximal section of the core.

Clause 19. The intravascular device of clause 18, wherein the plurality of fenestrations form a plurality of axially extending beams and a plurality of circumferentially extending rings.

Clause 20. The intravascular device of clause 18 or clause 19, wherein the stepped shoulder has an outer diameter that is 30% to 90% of the outer diameter of the proximal section of the core.

### Additional Terms & Definitions

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may also include properties and/or features (e.g., ingredients, components, members, elements, parts, and/or portions) described in one or more separate embodiments and are not necessarily limited strictly to the features expressly described for that particular embodiment. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

1. An intravascular device, comprising:
a core having a proximal section and a distal section, wherein the core includes a stepped shoulder at a transition from the proximal section to the distal section, the stepped shoulder having a constant outer diameter, the core forming a distally facing surface proximal of the stepped shoulder; and
a tube coupled to the core at the stepped shoulder, the distal section of the core passing into and being encompassed by the tube.

2. The intravascular device of claim 1, wherein the outer diameter of the stepped shoulder and an inner diameter of the tube are substantially similar.

3. The intravascular device of claim 1, wherein an outer diameter of the tube is substantially the same as an outer diameter of the proximal section of the core.

4. The intravascular device of claim 1, wherein an outer diameter of the tube is greater than an outer diameter of the proximal section of the core.

5. The intravascular device of claim 1, the device further comprising a hydrophilic coating.

6. The intravascular device of claim 1, wherein the tube is formed from a super-elastic material.

7. The intravascular device of claim 1, wherein at least a portion of the distal section of the core is a flat ribbon.

8. The intravascular device of claim 1, wherein the core is formed from stainless steel.

9. The intravascular device of claim 1, further comprising a radiopaque element disposed at or near a distal end of the device.

10. The intravascular device of claim 1, wherein the tube includes a plurality of fenestrations.

11. The intravascular device of claim 10, wherein the plurality of fenestrations form a plurality of axially extending beams and a plurality of circumferentially extending rings.

12. The intravascular device of claim 1, wherein the stepped shoulder has a length of about 1 cm to about 20 cm.

13. The intravascular device of claim 1, wherein the distally facing surface is perpendicular to a longitudinal axis of the core.

14. The intravascular device of claim 1, wherein the tube abuts the distally facing surface.

15. The intravascular device of claim 1, wherein the distal section of the core further comprises one or more tapered shoulders distal of the stepped shoulder.

16. The intravascular device of claim 1, further comprising one or more coils disposed between the core and the tube.

17. The intravascular device of claim 1, wherein the stepped shoulder has an outer diameter that is 30% to 90% of the outer diameter of the proximal section of the core.

18. An intravascular device, comprising:
a core having a proximal section and a distal section, wherein the core includes a stepped shoulder at a transition from the proximal section to the distal section, the stepped shoulder having a constant outer diameter, the core forming a distally facing surface adjacent to and proximal of the stepped shoulder, wherein the distally facing surface is perpendicular to a longitudinal axis of the core; and
a tube coupled to the core at the stepped shoulder, the distal section of the core passing into and being encompassed by the tube, and the tube abutting the distally facing surface,
wherein the outer diameter of the stepped shoulder and an inner diameter of the tube are substantially similar,
wherein an outer diameter of the tube is substantially the same as an outer diameter of the proximal section of the core.

19. The intravascular device of claim 18, wherein the tube comprises a plurality of fenestrations that form axially extending beams and circumferentially extending rings.

20. The intravascular device of claim 18, wherein the stepped shoulder has an outer diameter that is 30% to 90% of the outer diameter of the proximal section of the core.
